(19) United States
(12) Patent Application Publication
(10) Pub. No.: US 2013/0046267 A1
(43) Pub. Date: Feb. 21, 2013

(54) Diaper

(71) Applicant: William James Brinkley, II, Richmond, TX (US)

(72) Inventor: William James Brinkley, II, Richmond, TX (US)

(21) Appl. No.: 13/211,222

(22) Filed: Aug. 16, 2011

(51) Int. Cl. A61F 13/49

(52) U.S. Cl. 604/385.101

(57) ABSTRACT

This disclosure relates to an improved diaper. In one embodiment, a diaper can comprise a ring layer having an adhesive on a top portion of said ring layer. The diaper can also comprise a surface layer comprising an orifice connecting to said orifice is surrounded by said ring layer. Furthermore, the diaper can comprise an outer impermeable barrier connecting to said surface layer and extend from a top portion to a back portion of said diaper, and an absorbent layer between said surface layer and said outer impermeable barrier.

### BACKGROUND

This disclosure relates to an improved diaper capable of isolating human waste from skin.

Nowadays, diapers are worn by both infants who are not potty trained yet and by adults who suffer from involuntary release. When diapers are soiled, they must be changed promptly. If the soiled diapers are not changed on time and on a regular basis, skin disorders such as contact dermatitis and candida yeast infection can develop due to the feces and urine trapped inside the diaper against the skin. Since risk of infection is higher for babies, it is advisable to change diapers often. However, a baby can urinate or defecate as many as 5-7 times per day, irregularly. Consequentially, a parent may miss an incident, and a baby can go an extended period of time before being changed.

As such it would be useful to have an improved diaper capable of isolating human waste from the skin.

### SUMMARY

This disclosure relates to an improved diaper. In one embodiment, a diaper can comprise a ring layer having an adhesive on a top portion of said ring layer. The diaper can also comprise a surface layer comprising an orifice surrounded by said ring layer. Furthermore, the diaper can comprise an outer impermeable barrier connecting to said surface layer and extending from a top portion to a back portion of said diaper, and an absorbent layer between said surface layer and said outer impermeable barrier.

In another embodiment, the diaper can comprise a surface layer wherein said surface layer comprises a front portion and a back portion, an upper absorbent layer, a middle impermeable layer placed under said upper absorbent layer, and a lower absorbent layer. The diaper can also further comprise a bottom impermeable barrier placed below said lower absorbent layer, and an orifice wherein said orifice passing through said surface layer, said upper absorbent layer, and said middle impermeable layer.

Finally, in another embodiment, the diaper can comprise a front section comprising a first ring layer, a first surface layer, and an absorbent layer, further wherein said first surface layer comprises an orifice, further wherein said ring layer surrounds said orifice. Additionally, the diaper comprises a rear section comprising a second ring layer, a second surface layer, and second absorbent layer, further wherein said first surface layer comprises an orifice, further wherein said ring layer surrounds said orifice. Lastly, the diaper can comprise a bottom impermeable layer under said front section and said rear section.

### BRIEF DESCRIPTION OF THE DRAWINGS

FIG. 1 illustrates a diaper comprising of a ring layer, an absorbent layer, a one or more adhesive strips, and a one or more elastic elements.

FIG. 2 illustrates a side view of diaper with one absorbent layer.

FIG. 3 illustrates a perspective view of diaper that comprises ring layer.

FIG. 4 illustrates a cut view of another embodiment of diaper comprising surface layer, upper absorbent layer, middle impermeable layer, lower absorbent layer, and bottom impermeable barrier.

FIG. 5 illustrates a top view of another embodiment of a diaper.

FIG. 6 illustrates a cut view of diaper with a front chamber and a second chamber.

### DETAILED DESCRIPTION

Described herein is an improved diaper. The following description is presented to enable any person skilled in the art to make and use the invention as claimed and is provided in the context of the particular examples discussed below, variations of which will be readily apparent to those skilled in the art. In the interest of clarity, not all features of an actual implementation are described in this specification. It will be appreciated that in the development of any such actual implementation (as in any development project), design decisions must be made to achieve the designers' specific goals (e.g., compliance with system- and business-related constraints), and that these goals will vary from one implementation to another. It will also be appreciated that such development effort might be complex and time-consuming, but would nevertheless be a routine undertaking for those of ordinary skill in the field of the appropriate art having the benefit of this disclosure. Accordingly, the claims appended hereto are not intended to be limited by the disclosed embodiments, but are to be accorded their widest scope consistent with the principles and features disclosed herein.

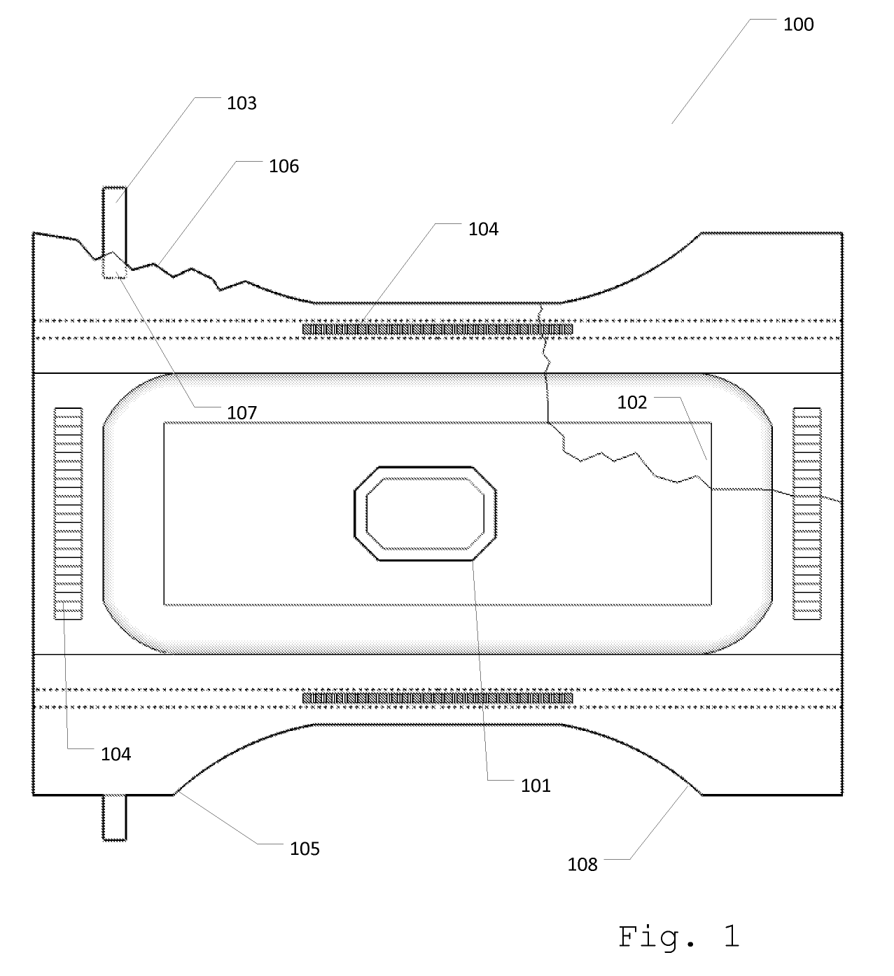

FIG. 1 illustrates a top view of a diaper 100 comprising a ring layer 101, an absorbent layer 102, a one or more adhesive strips 103, and a one or more elastic elements 104. Ring layer 101 can be laid onto the top of absorbent layer 102. An example of ring layer 101 can be, but is not limited to, a thin double-sided tape, dimensional double-sided tape, and/or a lingerie tape. Absorbent layer 102 is an absorbent containment material placed inside diaper 100 capable of capturing and retaining waste. Adhesive strips 103 can be in one embodiment an extrusion from diaper 100. In another embodiment, adhesive strips 103 can be a one or more strings sealed to a left pad 105 and a right pad 106 of diaper 100. In one embodiment wherein adhesive strip 103 is a string, adhesive strip 103 can be sown at a portion of the front side 107 of diaper 100. In another embodiment, adhesive strips 103 can be a hook-and-loop fastener with the hooks annexed to a portion of front side 107 of diaper 100, and loops affixed to a portion of a back side 108 of diaper 100. Elastic element 104 can be placed within the tissue on both left and right wing of diaper 100. In another embodiment, elastic element 104 can also be placed in the inner fragment of the front and back side of diaper 100. Elastic element 104 can provide a firm grip when diaper 100 is placed onto a user.

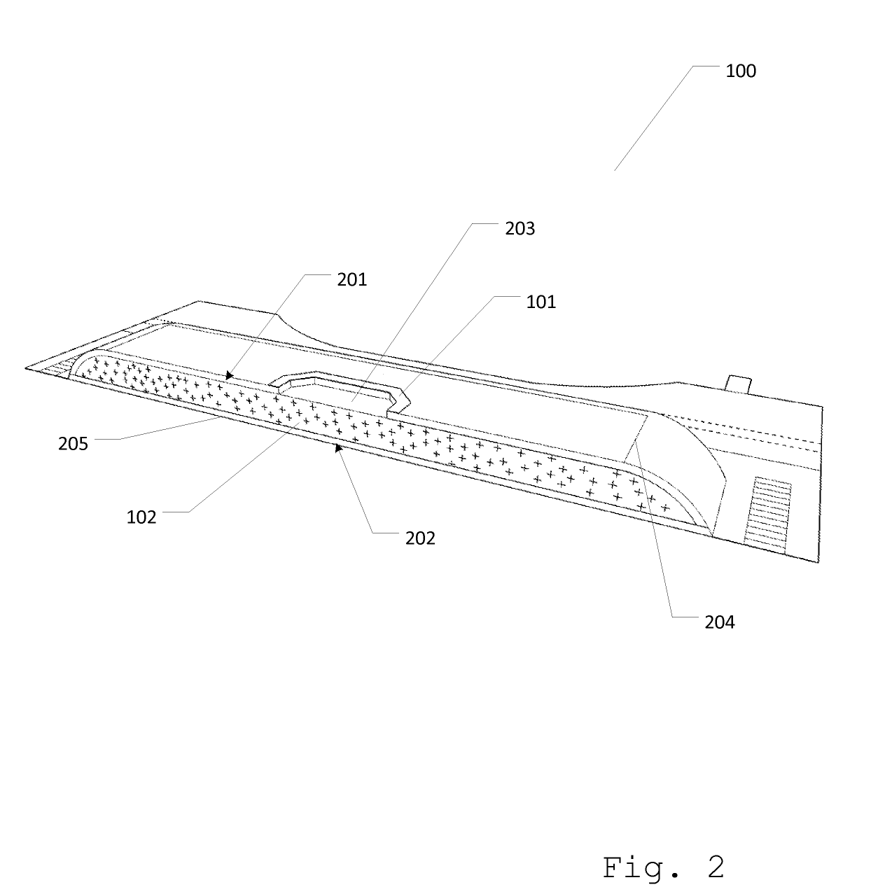

FIG. 2 illustrates a side view of diaper 100. In one embodiment, diaper 100 can comprise three separate layers: a surface layer 201, an absorbent layer 102 and an outer impermeable barrier 202. In one embodiment, surface layer 201 can be permeable. In another embodiment, a front portion of surface layer 201 can be permeable while the remaining portion can be impermeable. Surface layer 201 can comprise an orifice 203. Ring layer 101 can surround orifice 203. Orifice 203 can allow waste to flow to absorbent layer 102, keeping the waste away from the user's skin. Outer impermeable barrier 202 can prevent the waste from leaving diaper 100. Outer impermeable barrier 202 can be connected to surface layer 201 and can extend from a top portion 204 to a back portion 205 of diaper 100. Absorbent layer 102 can be in on embodiment a piece of absorption material that is capable of retaining waste. In another embodiment, absorbent layer 102 can also be small, scattered granules, or any other absorbent particulates known in the art. As waste enters orifice 203, absorbent layer 102 can push away from surface layer 201, creating a pocket for waste to stay. In one embodiment, orifice 203 can be positioned to capture urine. In another embodiment, orifice 203 can be positioned to capture feces. In another embodiment, diaper 100 can comprise two orifices 203 for the capture of both urine and feces.

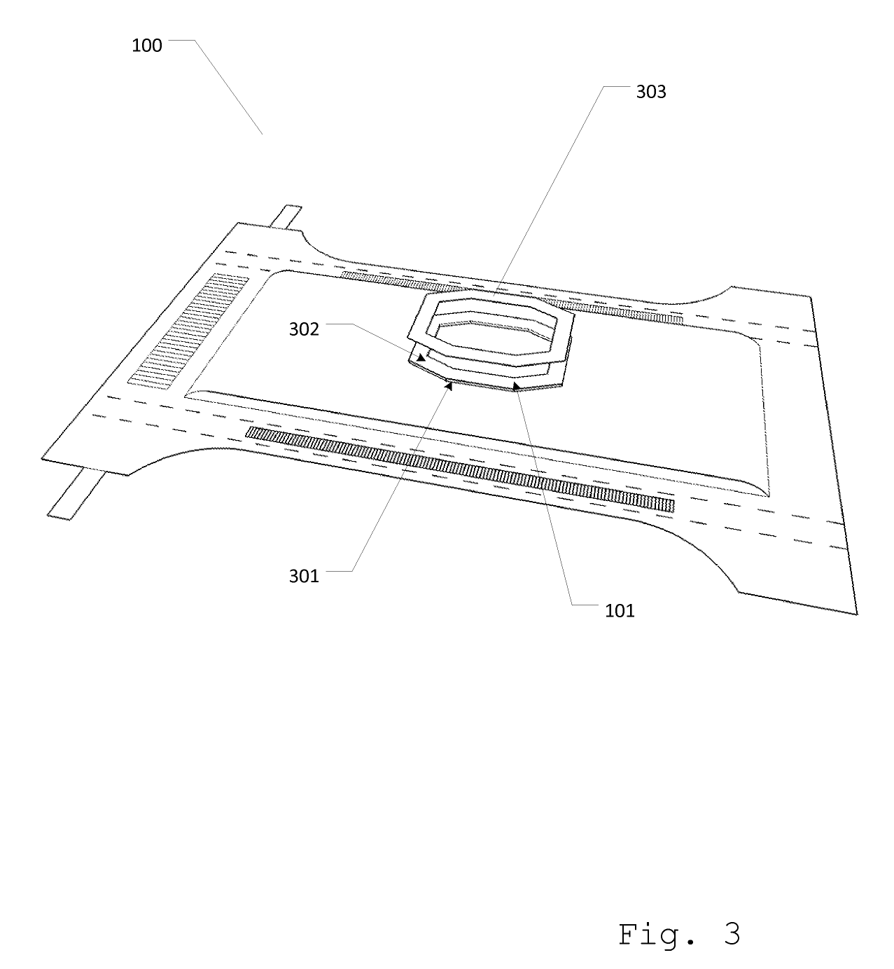

FIG. 3 illustrates a perspective view of diaper 100 that comprises ring layer 101. In one embodiment, ring layer 101 can comprise of two surfaces: a bottom surface 301 and a top surface 302. Top surface 302 can comprise an adhesive and a temporary adhesive cover 303. To affix ring layer 101 to buttocks appropriately, top adhesive surface 302 can be revealed by means of peeling off temporary adhesive cover 303, and, with sufficient pressure, pressing ring layer 101 firmly against the buttocks of a user, creating a seal to contain waste.

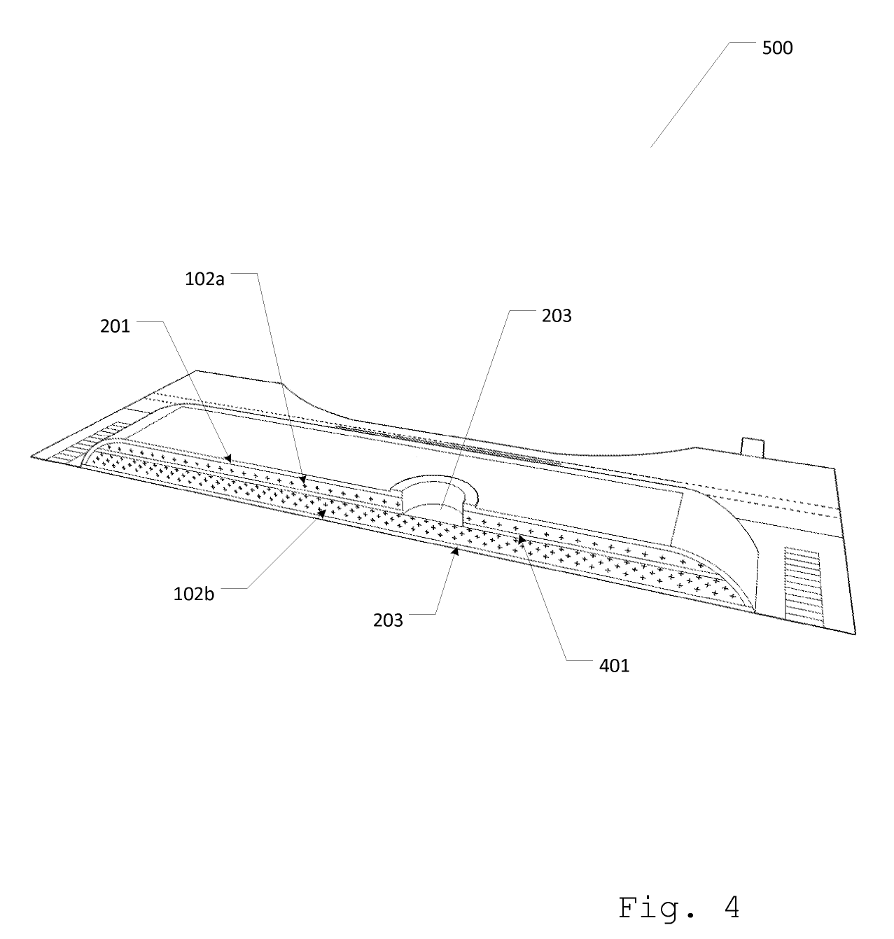

FIG. 4 illustrates a cut view of another embodiment of diaper 400 comprising surface layer 201, upper absorbent layer 102a, middle impermeable layer 401, lower absorbent layer 102b, and bottom impermeable barrier 203. In one embodiment, surface layer 201 can be permeable. In another embodiment, front portion of surface layer 201 can be permeable and back portion of surface layer 201 can be impermeable. In either embodiment, surface layer 201 permeable portion can be porous and permeable in such a way that urine can travel through the pores and can get absorbed into upper absorbent layer 102a. Middle impermeable layer 401 can be placed under upper absorbent layer 102a. Furthermore, middle impermeable layer 401 can be non-porous. Lower absorbent layer 102b can collect feces from the orifice 203. Orifice 203 passes through surface layer 201, upper absorbent layer 102a, and middle impermeable layer 401. As waste enters orifice 203, lower absorbent layer 102b can push away from middle impermeable layer 401 creating a pocket for waste to stay. Middle impermeable layer 401 can prevent feces from soaking through upper absorbent layer 102a and/or surface layer 201. In order to prevent feces from leaving lower absorbent layer 102b, bottom impermeable barrier 203 can be non-permeable.

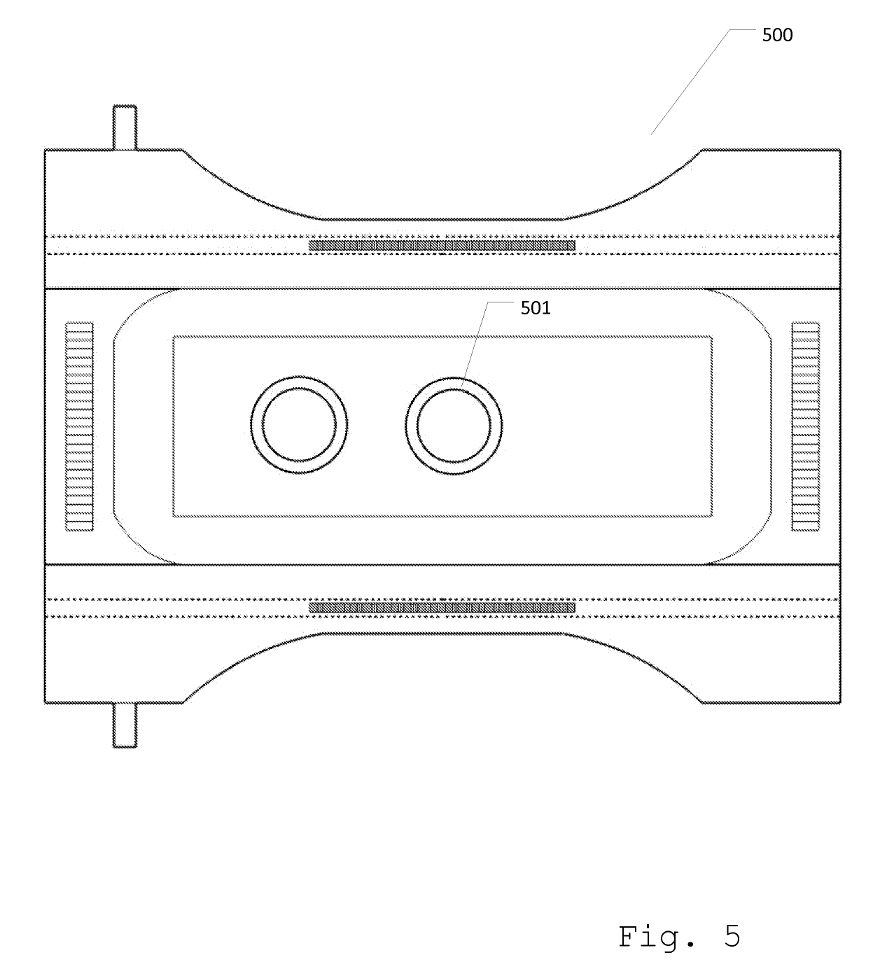

FIG. 5 illustrates a top view of another embodiment of diaper 100. In one embodiment, diaper 100 can comprise an additional orifice 203 specifically for isolating urine separately from feces material. The benefit of having an additional orifice 203 is to isolate urine further from the skin of a user.

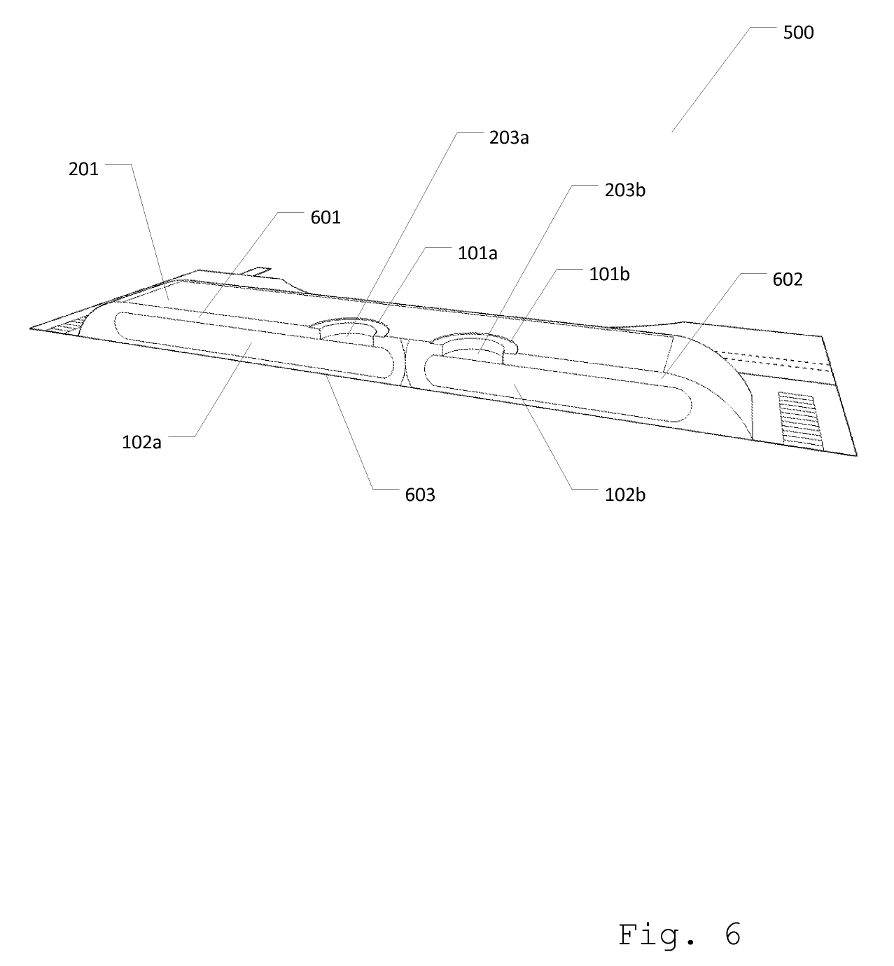

FIG. 6 illustrates a cut view of diaper 100. In such embodiment, diaper 100 can comprise a front section 601, a rear section 602, and a bottom impermeable layer 603. Front section 601 can be placed beside rear section 602. Front section 601 can comprise a first ring layer 101a, a surface layer 601, and a first absorbent layer 102a. Surface layer 201 can comprise a front orifice 203a. Front section 601 can collect urine. Rear section 602 can comprise a second ring layer 101b, surface layer 601, and second absorbent layer 102b. Surface layer 201 can comprise a rear orifice 203b. Rear section 602 can collect feces.

Various changes in the details of the illustrated operational system are possible without departing from the scope of the following claims. Some embodiments may combine the features described herein as being separate features. Similarly, one or more of the described features may be omitted, depending upon the specific operational environment the diaper is being implemented in. It is to be understood that the above description is intended to be illustrative, and not restrictive. For example, the above-described embodiments may be used in combination with each other. Many other embodiments will be apparent to those of skill in the art upon reviewing the above description. The scope of the disclosure should, therefore, be determined with reference to the appended claims, along with the full scope of equivalents to which such claims are entitled. In the appended claims, the terms “including” and “in which” are used as the plain-English equivalents of the respective terms “comprising” and “wherein.”

1. A diaper comprising
a ring layer comprising an adhesive on a top portion of said ring layer;
a surface layer comprising an orifice wherein said orifice is surrounded by said ring layer;
an outer impermeable barrier connecting to said surface layer and extending from a top portion to a back portion of said diaper; and,
an absorbent layer between said surface layer and said outer impermeable barrier.

2. The diaper of claim 1 wherein said ring layer is a thin double-sided tape.

3. The diaper of claim 1 wherein said ring layer is a dimensional double-sided tape.

4. The diaper of claim 1 wherein said ring layer is a lingerie tape.

5. The diaper of claim 1 wherein said surface layer comprises a second orifice in a front portion of said surface layer.

6. The diaper of claim 5 wherein said ring layer surrounds said second orifice.

7. A diaper comprising
a surface layer wherein said surface layer comprises a front portion and a back portion;
an upper absorbent layer;
a middle impermeable layer placed under said upper absorbent layer;
a lower absorbent layer;
a bottom impermeable barrier placed below said lower absorbent layer; and
an orifice wherein said orifice passes through said surface layer, said upper absorbent layer, and said middle impermeable layer.

8. The diaper of claim 7 wherein said front portion is permeable.

9. The diaper of claim 7 wherein said back portion is impermeable.

10. The diaper of claim 7 further comprising a ring layer, wherein said ring layer surrounds said orifice, further wherein said ring layer comprises an adhesive on a top portion of said ring layer.

11. The diaper of claim 10 wherein said ring layer comprises an adhesive.

12. The diaper of claim 7 further comprising a second orifice wherein sad second orifice passes through said surface layer.

13. The diaper of claim 6 wherein said bottom impermeable barrier is non-permeable.

14. A diaper comprising
a front section comprising a first ring layer, a first portion of a surface layer, and an absorbent layer, further wherein said first portion of said surface layer comprises an orifice, further wherein said ring layer surrounds said orifice;
a rear section comprising a second ring layer, a second portion of said surface layer, and a second absorbent layer, further wherein said second portion of said surface layer comprises an orifice, further wherein said ring layer surrounds said orifice; and
a bottom impermeable layer under said front section and said rear section.

15. The diaper of claim 14 further comprising an impermeable barrier between said front section and said second section.

16.
